(19) United States
(12) Patent Application Publication
(10) Pub. No.: US 2008/0089918 A1
(43) Pub. Date: Apr. 17, 2008

(54) Viscoelastic Solutions Containing Sodium Hyaluronate And Hydroxypropyl Methyl Cellulose, Preparation And Uses

(75) Inventor: Pierre Lebreton, Annecy Le Vieux (FR)

Correspondence Address: COHEN, PONTANI, LIEBERMAN & PAVANE, 551 FIFTH AVENUE, SUITE 1210, NEW YORK, NY, 10176, US

(73) Assignee: Comeal Industrie, Pringy (FR)

(21) Appl. No.: 11/791,603

(22) PCT Filed: Nov. 28, 2005
(86) PCT No.: PCT/FR05/50996

§ 371 Date: Dec. 10, 2007

(30) Foreign Application Priority Data
Nov. 30, 2004 (FR) 0412662

(51) Int. Cl. A61K 31/717; A61F 2/00

(52) U.S. Cl. 424/423; 514/57

(57) ABSTRACT

The present invention relates to: biocompatible, viscoelastic aqueous solutions based on a mixture of sodium hyaluronate(s) (NaHA) and hydroxypropyl methyl cellulose(s) (HPMC), which contain:
from 1 to 2% by weight of at least one sodium hyaluronate having an average molecular weight of between 1.10⁶ g/mol and 3.5.10⁶ g/mol; and from 0.2 to 1% by weight of at least one hydroxypropyl methyl cellulose having an average molecular weight of between 10,000 g/mol and 110,000 g/mol; the preparation of said solutions; and their use as surgical auxiliaries and/or temporary implants and as hydration implants.

The present invention relates to novel viscoelastic solutions containing sodium hyaluronate (NaHA) and hydroxypropyl methyl cellulose (HPMC). They are biocompatible aqueous solutions that are very particularly suitable as surgical auxiliaries and/or temporary implants. The present invention further relates to the preparation and use of said solutions as surgical auxiliaries and/or temporary implants and as hydration implants.

Every surgical invasion causes tissue damage. To minimize the damage involved, especially in areas where the tissues are particularly fragile and/or irreplaceable, it is known to use viscoelastic solutions as surgical auxiliaries. Such solutions protect the tissues from the surgical instruments and assist the manipulation of said tissues. They are also used for maintaining spaces or volumes to prevent tissues from coalescing and destroying such spaces or volumes. Solutions of this type are used very particularly in ophthalmic surgery and more specifically in cataract surgery.

With reference to said cataract surgery, the following commercial products have thus already been proposed:
Viscoat®, from Alcon Surgical, Inc., which contains sodium hyaluronate (NaHA) and chondroitin sulfate; this product is currently the market leader;
Healon® and Healon GV®, currently marketed by A.M.O., Amvisc® and Amvisc Plus®, currently marketed by Bausch & Lomb, Vitrax®, currently marketed by A.M.O., and Viscornéal® and Biocornéal®, marketed by the Applicant, which contain sodium hyaluronate (NaHA);
Orcolon®, from Optical Radiation Corporation, which contained a polyacrylamide and is now unavailable; and
Occucoat®, from Storz, which contains hydroxypropyl methyl cellulose (HPMC).

Each of these commercial products has advantages and disadvantages. Thus, Viscoat® is very efficient for adhering and protecting the tissues, especially the corneal endothelium. However, when the intervention has finished, it is difficult to remove this product from the anterior chamber and other products are more efficient than Viscoat® for the insertion of intraocular lenses.

Products of the same type have also been described:
in patent application WO-A-95 07085. More precisely, said document describes ophthalmic solutions of modified mucopolysaccharide which contain a viscoelastic fraction consisting of hyaluronic acid substituted by acyl groups having 3 to 20 carbon atoms, hyaluronic acid (HA), hydroxypropyl methyl cellulose (HPMC) or mixtures of these compounds. Said fraction does not contain chondroitin sulfate. Solutions of HA (of low or high molecular weight) and HPMC (of low or high molecular weight), and more precisely solutions of this type containing 2% by weight of HPMC and 1% by weight of HA, are described. These solutions were prepared and tested on the laboratory scale;
in patent application WO-A-96 32929. More precisely, said document describes solutions containing a viscous or viscoelastic substance in an aqueous vehicle of controlled pH and osmolality. Said substance can be selected from hyaluronic acid or one of its salts and mixtures of hyaluronic acid or one of its salts with modified cellulose or modified collagen. The hyaluronic acid or one of its salts is present in an amount of 0.1 to 5% and has a molecular weight of between 0.5.10⁶ and 2.5.10⁶; the modified cellulose or collagen is also present in an amount of 0.1 to 5%. The solutions described are not truly identified (no precision is provided for the molecular weight of the modified collagen or modified cellulose that may be used (the inventor has demonstrated the importance of the molecular weight of hydroxypropyl methyl cellulose on the qualifies, especially optical qualities, of the solutions of the invention (cf. Example 5 below)), no information is given about the true nature of the associated compounds in Examples 3 to 6, and no information is given about the mode of preparation and method of purification of the products) and they were evidently prepared and tested only on the laboratory scale.

Furthermore, WO-A-03 059391 describes a surgical method during which the following are used in succession:
a viscoelastic agent based on a first ingredient (hyaluronate), and then
a very fluid irrigating solution containing a second ingredient. Solutions incorporating both said ingredients are not proposed for use in said surgical method.

The mixtures tested in the Examples, which are representative of mixtures produced in situ (inside the eye), are not solutions in terms of the invention (the irrigating solution only modifies the surface properties of the viscoelastic agent). The mixtures produced in situ are not homogeneous mixtures and, furthermore, microbubbles are inexorably generated a priori at the viscoelastic agent/irrigating solution interface.

The teaching of WO-A-03 059391 neither anticipates nor suggests the subject of the present invention (ready-to-use solutions characterized by the nature of their constituents, their concentrations and their respective molecular weights (cf. below)). On the contrary, it can objectively be considered to distance those skilled in the art from said subject. Said teaching, namely the successive use of two ingredients, is far from suggesting the possibility of prepreparing (industrially) solutions that incorporate both these types of ingredients.

In such a context, the invention proposes novel viscoelastic solutions which are efficient in terms of viscosity, elasticity, adhesion, spreading and covering of the tissues, and which can be obtained with a high optical quality under advantageous industrial conditions.

The solutions of the invention are biocompatible, viscoelastic aqueous solutions based on a mixture of sodium hyaluronate(s) (NaHA) and hydroxypropyl methyl cellulose(s) (HPMC). They actually contain at least one sodium hyaluronate (NaHA) of a given average molecular weight and at least one hydroxypropyl methyl cellulose (HPMC) of a given average molecular weight; they generally contain sodium hyaluronate (NaHA) of a given average molecular weight and hydroxypropyl methyl cellulose (HPMC) of a given average molecular weight.

Characteristically, the biocompatible, viscoelastic aqueous solutions of the invention contain:
from 1 to 2% by weight, advantageously from more than 1 to 2% by weight, of at least one sodium hyaluronate having an average molecular weight of between 1.10⁶ g/mol and 3.5.10⁶ g/mol; and
from 0.2 to 1% by weight, advantageously from 0.2 to less than 1% by weight, of at least one hydroxypropyl methyl cellulose having an average molecular weight of between 10,000 g/mol and 110,000 g/mol.

The sodium hyaluronate (NaHA) is used mainly in respect of the rheological properties, whereas the hydroxypropyl methyl cellulose (HPMC) is used mainly in respect of the surface properties.

Totally unexpectedly, within the solutions of the invention:
said HPMC develops said surface properties when used at a low concentration (≦1% by weight, advantageously <1% by weight) and with a low molecular weight (≦110,000 g/mol). The use of such a low-molecular HPMC at a low concentration makes the solutions of the invention easier to obtain on the industrial scale. The problems of filtration and optical quality of the product (transparency, absence of bubbles) remain completely manageable under these conditions;
with such an HPMC a synergistic effect is observed on the viscosity. This is of particular value in a context where the solutions are used to maintain spaces or volumes. In fact, the more viscous the product, the less one needs to use in order to fill a volume. The less one uses, the less there will remain in the eye after the intervention. Those skilled in the art are not unaware of the risks inherent in not evacuating all the product introduced into the eye, especially the risk of raising the intraocular pressure.

The advantageous variants above can be considered independently of one another and, advantageously, in combination with one another.

The valuable properties of the compositions of the invention are shown in the Examples below.

Preferably, the viscoelastic aqueous solutions of the invention contain:
from 1.2 to 1.8% by weight of at least one sodium hyaluronate having an average molecular weight of between 1.6.10⁶ g/mol and 3.10⁶ g/mol; and
from 0.35 to 0.8% by weight of at least one hydroxypropyl methyl cellulose having an average molecular weight of between 10,000 g/mol and 50,000 g/mol.

In general terms, and hence also within the framework of this preferred variant, the highest molecular weights are advantageously associated with the lowest concentrations and vice-versa (thus it is advantageous to use an NaHA at a concentration of 1.2% and with an average molecular weight of about 3.10⁶ g/mol or an NaHA at a concentration of 1.8% and with an average molecular weight of about 1.6.10⁶ g/mol).

It is pointed out here, for whatever purpose it may serve, that the solutions of the invention are entirely capable of containing at least two sodium hyaluronates of different average molecular weights (of between 1.10⁶ g/mol and 3.5.10⁶ g/mol) and/or at least two hydroxypropyl methyl celluloses of different average molecular weights (of between 10,000 g/mol and 110,000 g/mol). However, they generally contain one sodium hyaluronate of adequate molecular weight (of between 1.10⁶ g/mol and 3.5.10⁶ g/mol, advantageously of between 1.6.10⁶/mol and 3.10⁶ g/mol) and one hydroxypropyl methyl cellulose of adequate molecular weight (of between 10,000 g/mol and 110,000 g/mol, advantageously of between 10,000 g/mol and 50,000 g/mol).

Within the framework of advantageous variants, the solutions of the invention contain:

1.2% by weight of a sodium hyaluronate having an average molecular weight of about 3.106 g/mol; or

1.37% by weight of at least one sodium hyaluronate having an average molecular weight of between 2.10⁶ and 3.10⁶ g/mol; or

1.8% by weight of a sodium hyaluronate having an average molecular weight of about 1.6.10⁶ g/ml.

Within the framework of another advantageous variant, to be considered independently of or in combination with the above advantageous variants, the solutions of the invention contain 0.57% by weight of a hydroxypropyl methyl cellulose having an average molecular weight of about 20,000 g/mol.

The biocompatible solutions of the invention are advantageously buffered at physiological pH (pH 7). The buffer in question is advantageously a phosphate buffer.

The following procedure is recommended for the preparation of the solutions of the invention:
prepare purified sodium hyaluronate of adequate molecular weight(s);
prepare an adequate purified solution (adequate with respect to the concentration) of hydroxypropyl methyl cellulose(s) of adequate molecular weight(s);
dissolve an adequate amount (adequate with respect to the concentration) of said purified sodium hyaluronate in said solution;
homogenize the resulting solution; and
remove any agglomerates from the resulting solution by filtration.

Such a process constitutes the second subject of the present invention.

The sodium hyaluronate (having an adequate average molecular weight of between 1.10⁶ g/mol and 3.5.10⁶ g/mol or at least two adequate average molecular weights of between 1.10⁶ g/mol and 3.5.10⁶ g/mol) is generally present in the form of fibers, whereas the hydroxypropyl methyl cellulose (having an adequate average molecular weight of between 10,000 g/mol and 110,000 g/mol or at least two adequate average molecular weights of between 10,000 g/mol and 110,000 g/mol) is generally present in powder form.

The purifications are performed independently of one another within the framework of the process of the invention. Advantageous variants for carrying out said purifications, which are particularly suited to the nature of the products in question, are specified below.

The step for filtration of the mixture is advantageously performed on at least one 5 μm filter.

Whatever the case may be, this filtration step can be carried out under reasonable conditions and within a reasonable time in view of the low concentration and molecular weight of the hydroxypropyl methyl cellulose(s) present in the solution.

To obtain the purified sodium hyaluronate, it is recommended to carry out the purification upstream because of the high viscosity of the solutions containing this product. Advantageously, the following procedure is recommended.

Fibers of NaHA (of adequate molecular weight(s)) are dissolved at a low concentration. The dilute solutions obtained are filtered on increasingly fine filters. It is recommended in particular to filter them successively on filters of 5 μm, 1 μm and then 0.22 μm.

The purified solutions are then treated so that the purified NaHA precipitates out. The precipitation is generally carried out in alcohol. Fibers of purified NaHA are recovered after drying. Those skilled in the art are familiar with this process for the purification of sodium hyaluronate.

It is difficult to apply a process of this type to hydroxypropyl methyl cellulose which does not have the same solubility characteristics as the sodium hyaluronate. Precipitation requires heating, which, in an industrial environment, can cause a proliferation of bacteria that are a source of endotoxins.

In addition, this purification process does nothing to solve the phenomena of opalescence.

For the preparation of purified solutions of hydroxypropyl methyl cellulose (precursor solutions), the recommended procedure according to the invention is filtration. The starting material is dissolved and the solution obtained is homogenized and then filtered. It actually undergoes successive filtrations on increasingly fine filters. It is pointed out here that such solutions are difficult to filter and that, quite obviously, the difficulty increases with the concentration of hydroxypropyl methyl cellulose.

It is recommended to filter successively on filters of 5 μm, 1 μm and then 0.22 μm.

Thus, particularly advantageously, the solutions of the invention are obtained as follows:
the NaHA starting material (fibers) has first been purified (by filtration of a dilute solution thereof on filters of 5 μm, 1 μm and then 0.22 μm);
the HPMC starting material (powder) has been dissolved in an adequate aqueous solution, said solution being successively filtered on filters of 5 μm, 1 μm and then 0.22 μm;
the purified NaHA is added in an adequate amount to the filtered solution and the latter is homogenized; and
the homogenized solution is finally filtered on a 5 μm filter.

This process for the preparation of the solutions of the invention was carried out especially to prepare the solutions of the Examples below.

Furthermore, the quality of the solution obtained can advantageously be improved by degassing said solution. Such degassing is intended to remove the small gas bubbles which the resulting viscoelastic solution may contain.

The solution obtained is then generally packaged and subsequently sterilized. It is generally packaged in syringes.

It is noted here, incidentally, that the sterilization performed may somewhat modify the molecular weights of the NaHA and HPMC present in the solution.

According to its third subject, the present invention relates to the use of the solutions of the invention as surgical auxiliaries and/or temporary implants and as hydration implants.

Surgical auxiliaries and/or temporary implants were referred to in the introduction to the present text. The solutions of the invention are particularly efficient in the context of this use and hence are particularly efficient in the context of cataract surgery. In the context of such a use, the solutions of the invention are injected, perform their function and are then recovered at the end of the intervention.

The opportunities for using said solutions are not limited to this context. They are also perfectly suitable as hydration implants, e.g. in the context of a mesolift. Such hydration implants are injected, perform their function and disappear at their injection site, where they are metabolized.

The solutions of the invention are generally used as such, but it is not excluded to incorporate at least one additive therein or for them to be laden with at least one active principle. The implants (surgical or hydration implants) and/or auxiliaries mentioned above therefore consist or consist essentially of the solutions of the invention.

It is now proposed to illustrate the invention and to emphasize its value by means of the Examples below.

Examples 1, 2 and 3 show the above-stated synergistic effect on the viscosity. The dynamic viscosity of the gels tested was measured with a CARIMED CSL 500 controlled stress rheometer (from TA Instruments) at a temperature of 25° C. using a cone-and-plate measuring device (4 cm, 2°). The dynamic viscosity at rest is determined by a measurement at equilibrium under a stress of 1 Pa.

Example 4 illustrates the problems encountered with the filtration of HPMC solutions.

Example 5 shows the importance of the concentration and molecular weight parameters of the HPMC used on the optical quality of the solution.

### EXAMPLE 1

Three aqueous solutions were prepared from the following ingredients:
NaHA fibers of molecular weight Mw≈2.5.10⁶ g/mol;
HPMC powder marketed by DOW and known by the name Methocel under the reference E4M, of molecular weight Mw≈86.10³ g/mol.

The first solution, containing the NaHA at a concentration of 1.28% by weight, had a viscosity at rest of 234±10 Pa·s.

The second solution, containing the HPMC at a concentration of 2% by weight (below this concentration, the solutions have such a low viscosity that it cannot be measured by the method employed), had a viscosity at rest of 4 Pa·s.

The third solution, namely the solution of the invention containing the same NaHA at a concentration of 1.28% by weight and the same HPMC at a concentration of 0.32%, had a viscosity at rest of 417±12 Pa·s. A variation of +183 Pa·s is therefore observed, whereas HPMC normally makes only a small contribution to the viscosity (cf. the 4 Pa·s stated above for a concentration approximately 6 times higher).

### EXAMPLE 2

Three aqueous solutions were likewise prepared from the following ingredients:
NaHA fibers of molecular weight Mw≈3.10⁶ g/mol;
HPMC powder marketed by Dow and known by the name Methocel under the reference E50, of molecular weight Mw≈20.103 g/mol.

The first solution, containing the NaHA at a concentration of 1.21% by weight, had a viscosity at rest of 213±9 Pa·s.

The second solution, containing the HPMC at a concentration of 2% by weight, had a viscosity at rest of 0.05 Pa·s.

The third solution, namely the solution of the invention containing the same NaHA at a concentration of 1.21% by weight and the same HPMC at a concentration of 0.47% by weight, had a viscosity at rest of 234±8 Pa·s. The increase in viscosity observed in this case is still greater than expected (synergy), but is more moderate than that observed in the previous Example.

### EXAMPLE 3

Three aqueous solutions were likewise prepared from the following ingredients:
NaHA fibers of molecular weight Mw≈2.5.106 g/mol;
HPMC powder marketed by DOW and known by the name Methocel under the reference E50, of molecular weight Mw≈20,000 g/mol.

The first solution, containing the NaHA at a concentration of 1.5% by weight, had a viscosity at rest of 161±2 Pa·s.

The second solution, containing the HPMC at a concentration of 2% by weight, had a viscosity at rest of 0.05 Pa·s.

The third solution, namely the solution of the invention containing the same NaHA at a concentration of 1.5% by weight and the same HPMC at a concentration of 0.57% by weight, had a viscosity at rest of 185±7 Pa·s. The synergistic effect is still observed in this context.

### EXAMPLE 4

Homogeneous solutions of HPMC are obtained by dissolving adequate amounts of powders (Methocel marketed by DOW) in phosphate buffer solutions.

Homogenization is effected by mechanical agitation at room temperature. After 48 h of agitation, the solutions are purified by successive filtrations culminating in filtration on 0.2 μm filters.

The quantity of solution filtered was measured as a function of time. The results obtained with HPMCs of different molecular weights and/or at different concentrations are indicated in the Table below.

| HPMC | Concentration | Type of filtration | Quantity/time |
|---|---|---|---|
| HPMC | 0.57% | 0.2 μm filter | 4000 ml in 10 min |
| 20,000 g/mol | | | |
| HPMC | 2.0% | 1.2 μm filter | 115 ml in 20 min |
| 20,000 g/mol | | | |
| HPMC | 1.5% | 1.2 μm filter | 2 ml in 30 min |
| 86,000 g/mol | | | |

The above figures confirm that the higher the concentration of HPMC and/or the molecular weight of HPMC, the more difficult is the filtration. The economic value of the invention is obvious.

It is noted, incidentally, that the final filtration operation should actually be carried out, within the framework of the invention, on solutions containing the NaHA as well as the HPMC.

### EXAMPLE 5

A solution of the invention containing the following was prepared:

1.2% by weight of NaHA of Mw≈3.106 g/mol; and

1% by weight of HPMC of Mw≈1110,000 g/mol.

The solution exhibits opalescence phenomena that are still tolerable but inescapable.

Under these limiting conditions of the invention as regards the HPMC (upper limits of concentration and molecular weight), the product obtained is not of optimal quality. It is pointed out that said product is generally intended for use in opthalmology.

This demonstrates the great value of the solutions of the invention compared with those of the prior art described in documents WO-A-95 07085 and WO-A-96 32929, which generally have a higher concentration of HPMC and may have a greater molecular weight (prepared only on the laboratory scale).

1. A biocompatible, viscoelastic aqueous solution based on a mixture of sodium hyaluronate(s) (NaHA) and hydroxypropyl methyl cellulose(s) (HPMC), comprising:
from 1 to 2% by weight, advantageously from more than 1 to 2% by weight, of at least one sodium hyaluronate having an average molecular weight of between 1.10⁶ g/mol and 3.5.10⁶ g/mol; and
from 0.2 to 1% by weight, advantageously from 0.2 to less than 1% by weight, of at least one hydroxypropyl methyl cellulose having an average molecular weight of between 10,000 g/mol and 110,000 g/mol.

2. The solution according to claim 1, further comprising:
from 1.2 to 1.8% by weight of at least one sodium hyaluronate having an average molecular weight of between 1.6.10⁶ g/mol and 3.10⁶ g/mol; and
from 0.35 to 0.8% by weight of at least one hydroxypropyl methyl cellulose having an average molecular weight of between 10,000 g/mol and 50,000 g/mol.

3. The solution according to claim 1, further comprising it sodium hyaluronate of adequate molecular weight and hydroxypropyl methyl cellulose of adequate molecular weight.

4. The solution according to claim 1, further comprising 1.2% by weight of a sodium hyaluronate having an average molecular weight of about 3.10⁶ gμmol; or 1.37% by weight of at least one sodium hyaluronate having an average molecular weight of between 2.10⁶ and 3.10⁶ g/mol; or 1.8% by weight of a sodium hyaluronate having an average molecular weight of about 1.6.10⁶ g/ml.

5. The solution according to claim 1, further comprising 0.57% by weight of a hydroxypropyl methyl cellulose having an average molecular weight of about 20,000 g/mol.

6. The solution according to claim 1, wherein the solution is buffered at physiological pH.

7. The solution according to claim 6, wherein the solution is buffered with a phosphate buffer.

8. A process for the preparation of a solution according to claim 1, comprising the steps of:
preparation of purified sodium hyaluronate of adequate molecular weight(s);
preparation of an adequate purified solution of hydroxypropyl methyl cellulose(s) of adequate molecular weight(s);
dissolution of an adequate amount of said purified sodium hyaluronate in said purified solution; and
homogenization of the resulting solution; followed by
filtration of said resulting solution.

9. The process according to claim 8, wherein said preparation of purified sodium hyaluronate comprises dissolution of the adequate starting material at a low concentration, successive filtrations of the resulting solution on increasingly fine filters, and then precipitation of the dissolved starting material purified in this way.

10. The process according to claim 8, wherein said preparation of the purified solution of hydroxypropyl methyl cellulose(s) comprises successive filtrations on increasingly fine filters.

11. Surgical auxiliaries and/or temporary implants, comprising a solution according to claim 1 or a solution prepared according to claim 8.

12. Hydration implants, comprising a solution according to claim 1 or a solution prepared according to claim 8.
